(19) United States
(12) Patent Application Publication
(10) Pub. No.: US 2026/0015639 A1
(43) Pub. Date: Jan. 15, 2026

(54) SYSTEM AND METHODS FOR THE GENERATION OF DNA STRANDS AND THE QUERYING OF A DNA DATABASE

(71) Applicant: Seagate Technology LLC, Fremont, CA (US)

(72) Inventors: Gemma MENDONSA, Edina, MN (US); Riyan Alex MENDONSA, Edina, MN (US)

(21) Appl. No.: 18/771,838

(22) Filed: Jul. 12, 2024

(51) Int. Cl. C12P 19/34; C12Q 1/6818

(52) U.S. Cl. CPC C12P 19/34; C12Q 1/6818

(57) ABSTRACT

The present disclosure describes a system and methods for the generation of DNA strands and querying a DNA database rapidly and accurately. The method disclosed includes the generating at least one database single stranded DNA (ssDNA) strand, includes a plurality of functional oligonucleotides separated by introns. On one end of each intron there is a donor fluorophore and on the other end is an acceptor fluorophore. A query ssDNA strand is also generated that includes a series of complimentary oligonucleotides, which can hybridize with the functional oligonucleotides of the database ssDNA strand. Hybridization causes the intron regions of the database ssDNA strand to fold resulting in the donor fluorophore and acceptor fluorophore being placed in close proximity to one another. The close proximity enables Förster Resonance Energy Transfer (FRET) phenomena to occur, which is detected using a photodetector.

### TECHNICAL FIELD

This disclosure relates to a method for the generation of DNA strands and the querying of a DNA database.

### BACKGROUND

The generation of DNA strands is important throughout the medical and research fields. Additionally, one very useful application of DNA strand synthesis is for DNA data storage.

Current DNA strand generation generally relies upon the phosphoramidite method. This methodology generates oligonucleotide strands on a nucleotide-by-nucleotide bases. This methodology generally has a limit of 200-300 base pairs, and then these oligonucleotides can be conjugated to generate longer DNA strands. New methodologies of DNA synthesis, such as terminal deoxynucleotidyl transferase, are being explored; however, such methodologies are not as effective and require further development before they are ready for commercial deployment.

In the space of DNA data storage, currently DNA strands which have been synthesized are read, using known sequencing technologies, to determine which base pairs are located in which order to extract the stored information. These techniques, while being able to store massive amounts of information in a very dense manner, suffer from very slow reading times (as sequencing technologies are still relatively slow) and is also limited to sequential reads.

Due to the limitations on the generation of effectively generating longer DNA strands in an efficient manner, and further the inability to quickly read DNA strands, it is desirable to improve the speed of DNA strand synthesis and rapid DNA database querying.

### SUMMARY

The present disclosure describes a system and methods for the generation of DNA strands and querying a DNA database rapidly and accurately. Such systems and methods enable more rapid detection of target DNA segments in a sample, which has significant utility in the medical diagnostic and DNA storage fields.

In particular, the method disclosed includes the generating at least one database single stranded DNA (ssDNA) strand, wherein the database ssDNA strand is comprised of a plurality of functional oligonucleotides separated by introns. Each intron includes a 3′ end and a 5′ end. On one end of each intron there is a donor fluorophore and on the other end is an acceptor fluorophore.

A query ssDNA strand is also generated. The query ssDNA stand comprises a series of complimentary oligonucleotides, which are capable of hybridizing with the functional oligonucleotides the database ssDNA strand. The query ssDNA can be isolated from a biological sample, or may be synthetically derived.

The query ssDNA strand is introduced to the database ssDNA strand, which then hybridized (at the functional oligonucleotide regions). This hybridization causes the intron regions of the database ssDNA strand to fold resulting in the donor fluorophore on one end and the acceptor fluorophore on the other end of each intron to be placed in close proximity to one another. The close proximity of the donor and acceptor fluorophores enables Förster Resonance Energy Transfer (FRET) phenomena to occur at each intron. The emission of the acceptor fluorophore is detected using a photodetector.

In some embodiments, there is a plurality of database ssDNA strands which are unique from one another. These database ssDNA strands are immobilized on a substrate. The sequence for each database ssDNA strand is known, and the location on the substrate of each database ssDNA strand is known. Thus, when the FRET phenomena is detected, the sequence of the query ssDNA strand can be determined.

In some cases, the plurality of database ssDNA strands each correspond to a different pathogen's unique DNA, which allows the determination of which pathogen the query ssDNA strand is derived from. In other cases, the plurality of database ssDNA strands each correspond to a different antibiotics resistance DNA sequence, which allows the determination of which antibiotics a pathogen the query ssDNA strand is derived from is resistant to.

Also disclosed is a method for generating the database single stranded DNA (ssDNA) strands. This may be performed in order to generate a synthetic dataset for DNA storage purposes, as well as generating synthetic genomic or plasmid DNA. This synthetic DNA generation is done by receiving (or synthesizing) a plurality of functional oligonucleotides. Each end of the functional oligonucleotides includes a unique sequence of base pairs that are designed. A plurality of introns is also received or synthesized using a nucleotide-by-nucleotide process. Each end of the introns includes a unique sequence of base pairs that are designed, and each intron includes a donor fluorophore on one end and an acceptor fluorophore on the other end.

A plurality of DNAzymes is introduced to the plurality of functional oligonucleotides and the plurality of introns. The DNAzymes are designed to have ends that are of a known sequence. The designing the ends of the plurality of functional oligonucleotides, the plurality of introns, and the DNAzymes results in conjugation of the plurality of functional oligonucleotides and the plurality of introns in a desired order.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 is an example illustration of a DNAzyme coupling together two different DNA oligonucleotide strands.

FIG. 2 is an example illustration of a series of DNA strands that have been generated coupled to a substrate material.

FIG. 3A is an illustration of an example first workflow for the generation of a longer DNA strand using DNAzyme molecules.

FIGS. 3B and 3C are illustrations of an example second workflow for the generation of a longer DNA strand using DNAzyme molecules.

FIG. 4 is an illustration of a Forster Resonance Energy Transfer (FRET) phenomena.

FIG. 5 is an example of the fluorescence emission spectrum of molecules that exhibit FRET.

FIG. 6 is an illustration of an example workflow for the conjugation of a query DNA strand to a DNA library strand that causes a FRET phenomenon to occur.

FIG. 7 is an illustration of an example workflow for querying of a DNA database reliant upon a FRET phenomenon.

FIG. 8 is a flow diagram for an example first process of generating a DNA segment utilizing DNAzymes.

FIG. 9 is a flow diagram for an example second process of generating a DNA segment utilizing DNAzymes.

FIG. 10 is a flow diagram for an example process of querying a DNA database.

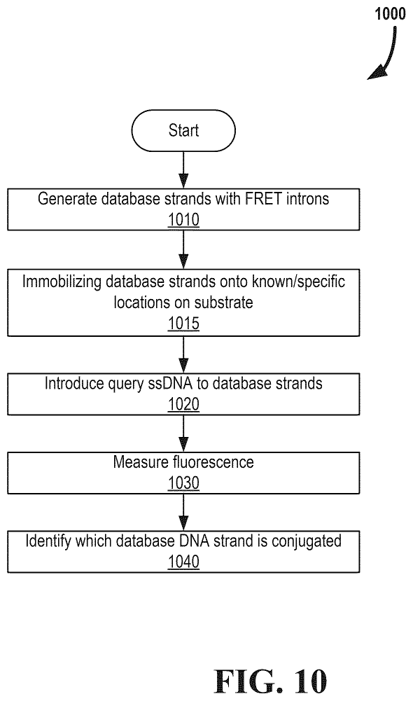

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

It should be noted that the terms “about”, “approximately”, “roughly” and the like may be employed. In general, these terms are intended to indicate that the value or range provided may vary by a set amount. Generally, the term “about” is intended to suggest a variation of plus or minus ten percent of the value provided. The terms “approximately” or “roughly” may indicate a value that is within one standard deviation of the provided value or range.

The present invention relates to both the generation of a DNA strand using DNAzymes as well as the ability to rapidly query a DNA database. Such methods have significant implication for the fields of medical diagnostics, research and DNA storage. In particular, the proposed systems allow for the rapid synthesis of long DNA strands from smaller oligonucleotide segments (“oligos”) and, conversely, the rapid detection of DNA segments, and when leveraged on a known database matrix, the ability of selectively identify the sequence of a query DNA strand (from among a plurality of known DNA database strands.

Focusing on the generation of longer DNA strands, the proposed systems rely upon the generation of initial oligonucleotide segments. These oligos generally are generated using known nucleotide-by-nucleotide synthesis techniques. These oligos are generally grown to the lengths of 100-300 base pairs in length. Beyond this length, standard nucleotide-by-nucleotide synthesis techniques begin to degrade. The key is to then couple these generated oligos together, in a selective manner, to generate longer strands of DNA. In the presently disclosed systems and methods, molecules known as DNAzymes may be employed to conjugate the oligos.

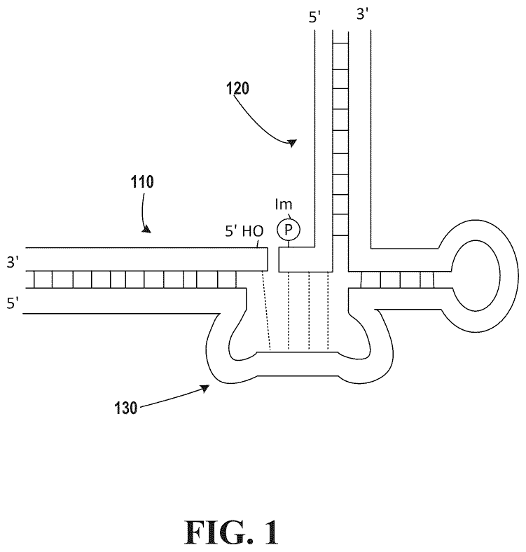

FIG. 1 provides an illustration of a DNAzyme 130 hybridized to the ends of two oligos 110 and 120, respectively. The DNAzyme 130 selectively hybridizes to 5′ end of one oligo 110 and 3′ of the other oligo 120. A DNAzyme 130 is a piece of DNA of a specific sequence that can fold into a structure that possesses catalytic activity, similar to how amino acids fold into a protein catalyst or enzyme. The instant DNAzyme 130 illustrated in FIG. 1 is the E47 DNAzyme reported in 1995. Each DNAzyme 130 has different sequences on the “ends”. These different sequences can only hybridize to corresponding oligo sequences, thereby ensuring the selectivity of the oligos that are ordered together. Thus, the ends of the oligos may be modified to ensure different sequences of oligos are conjoined. The folding portion of the DNAzyme 130 is not modifiable. The ligation of the oligos 110 and 120 by the DNAzyme 130 may be performed in the presence of zinc or copper ions. While the DNAzyme E47 is illustrated as being utilized herein, any known or discovered DNAzyme may be leveraged in the methods disclosed herein.

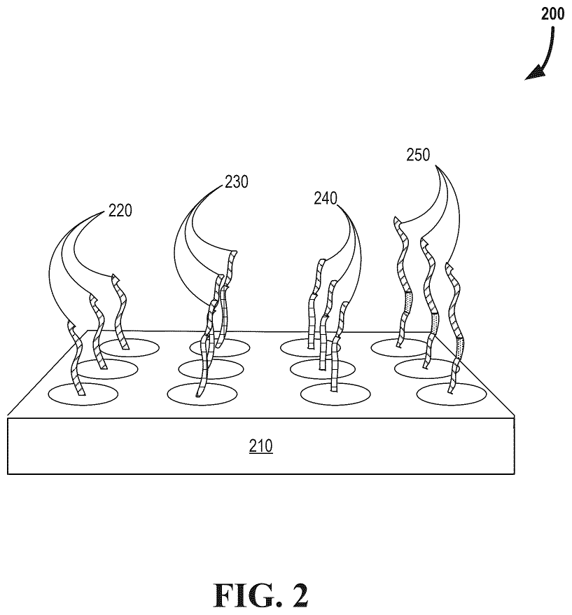

FIG. 2 provides an example illustration of a series of oligo and DNAzymes strands 220, 230, 240 and 250 respectively, that have been formed on a substrate 210. In some embodiments, the substrate 210 may be a silicon substrate. In other embodiments, the substrate 210 may be a lab-on-a-chip, column or any other appropriate medium.

The oligos and the DNAzymes may be generated using phophoramidite or enzymatic techniques. The oligos and the DNAzymes are designed such that their ‘ends’ are unique and complimentary. This allows particular strands 220, 230, 240 and 250 to self-assemble based upon the starting oligo that is bound to the substrate 210. Once the oligo-DNAzyme strands 220, 230, 240 and 250 have been assembled, they may be cleaved from the substrate 210, resulting in a solution with the DNAzyme-oligo strands 220, 230, 240 and 250. Cleavage may be performed using thermal, physical or chemical means.

Importantly, before assembly of the oligos, 3′ ends of each oligo may be required to be ‘activated’ by phosphate and imidazole prior to ligation. 1-Ethyl-3-[3-dimethylaminopropyl] carbodiimide hydrochloride (EDC or EDAC) is a zero-length crosslinking agent used to couple carboxyl groups to primary amines. EDC chemistry may be leveraged in this activation process.

In some embodiments, the EDC and imidazole may be removed from the solution after activation of the oligos and prior to the assembly of the oligo-DNAzyme strands. A reaction buffer (e.g., HEPES buffer with a neutral pH and specified salt content) and a heavy metal ion (e.g., Zn or Cu) is also added to the buffering solution. This solution activates the oligos and DNAzymes, causing the ligation. Again, self-assembly is controlled by the end sequences of the oligos and the DNAzymes. As such, based upon the design of the various oligos and the DNAzymes specific sequences/order of oligo strands may be generated. The oligo-DNAzymes strands may be purified from un-conjugated oligos and truncated assemblies. This purification may be performed prior to cleavage from the substrate, or alternatively post-cleavage.

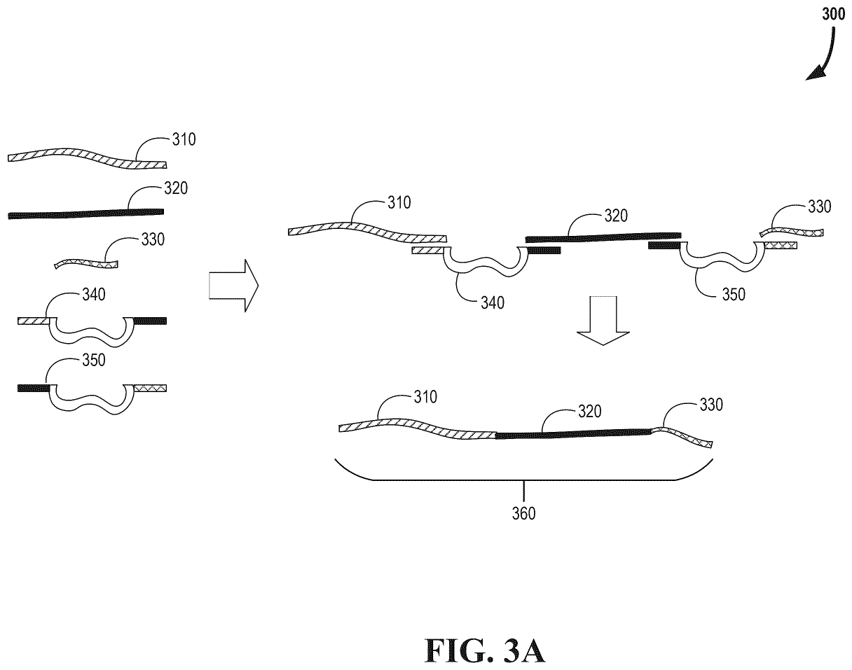

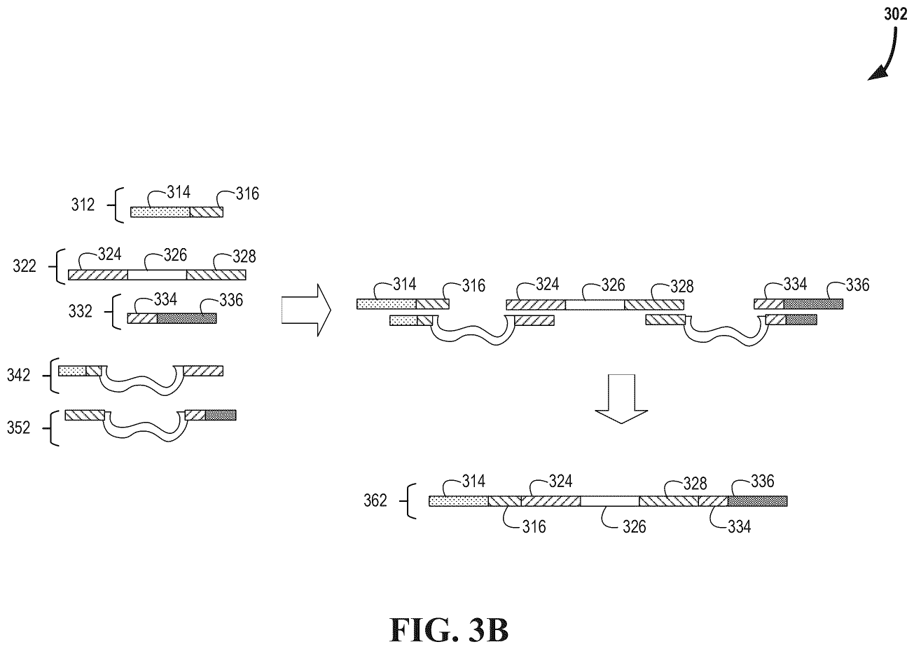

FIG. 3A provides a more detailed illustration of an example DNA strand 360 generation using different oligos 310, 320 and 330, respectively, based upon two distinctly designed DNAzymes 340 and 350, respectively. When combined in the reaction buffer, the 5′ end of the first oligo 310 matches the ‘end’ base pairs of the first DNAzyme 340. The other end of the DNAzyme 340 matches 3′ base pair sequence of the second oligo 320. Likewise, 5′ end of this second oligo 320 matches the base pairs of the modified sequence of the end of the second DNAzyme 350. The other end of the second DNAzyme 350 matches the sequence of 3′ side of the third oligo 330. Given these specifically designed DNAzymes 340 and 350 the order of the oligos 310, 320 and 330, respectively, is deterministic. The oligos 310, 320 and 330, respectively, are then ligated together by the DNAzymes 340 and 350 to generate the final single stranded DNA (ssDNA) lengthy segment 360. In this basic illustration, only three oligo are shown being ligated into a single ssDNA segment. This is purely for illustrative purposes however-much larger number of oligos may be ordered and ligated together in practice.

As such, one advantage of this method is that it is extremely scalable. As long as there are unique combinations of 3′ and 5′ ends of the oligos, extremely lengthy final ssDNA segments may be manufactured. It is also possible to perform multiple staged assembly processes. In this way subsections of a desired gene (or other synthetic sequence) may be synthesized in a single reaction, and other subsequences in another reaction. These different subsections may then be combined and joined via another DNAzyme reaction. This may be particularly advantageous where the same oligos are being utilized in each subsection but are being combined in a different order.

Synthesis and assembly steps may be performed using microfluidics, digital microfluidics, droplet levitation, inkjet dispensing, valve, pump or any other (or combined) liquid handling methodologies. For example, magnetic beads (or other charged particles) coupled to the various oligos may be employed to move the specific oligos to desired locations using magnetic or electrical fields.

While the foregoing DNAzyme reliant assembly process is focused upon DNA segments, it should be noted that these techniques may be utilized on other base pairs. For example, synthetic nucleotides, RNA or other types of nucleic acids may be used instead of ‘natural’ DNA nucleotides. It is also possible that an oligo strand may be phosphorylated by a protein or by a DNAzyme possessing the ability to phosphorylate the 3′ end of the oligonucleotide (such as 3′Kin1 DNAzyme).

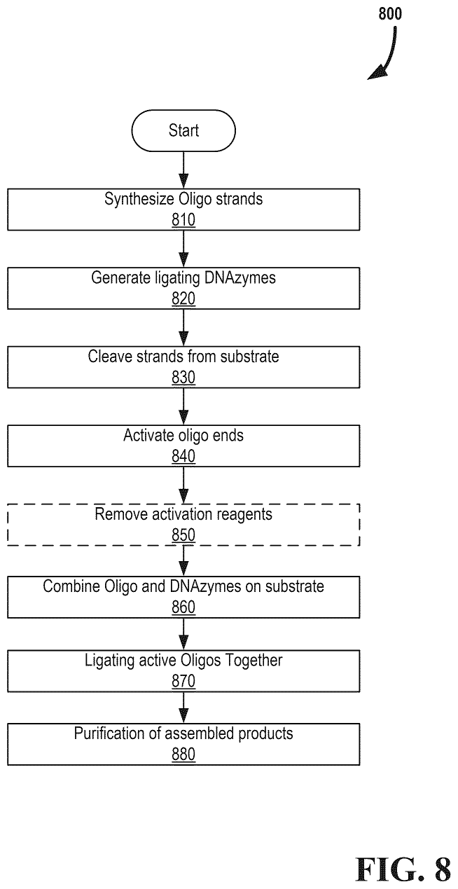

Turning to FIG. 8, the example process 800 of ssDNA segment generation form multiple conjugated oligonucleotides is provided. In the example process, the oligos are first generated (at 810). As previously noted, oligo synthesis may leverage phophoramidite or enzymatic techniques. The 3′ and 5′ ends of the oligos are designed in such a manner as to be unique between the various oligos for each segment reaction. Likewise, the DNAzymes are also generated (at 820) where the two ends of each DNAzyme are designed to ensure that the oligos, when hybridized to their respective DNAzymes, are properly ordered into the final strands. The oligo-DNAzyme complexes may then be cleaved from the substrate (at 830) when applicable. Again, cleavage may be chemical, thermal, or physical. The ends of the oligos are activated using EDC and imidazole (at 840) and this allows the ends of each oligo to conjugate to the neighboring oligo, thereby forming a ssDNA segment of particular order(s) of oligos. Optionally, the activation reagents may be removed from the working solution (at 850). The oligos and DNAzymes are combined (at 860), either in solution, or where the initial oligo is bound to a substrate (as may be advantageous in lab-on-a-chip or other microfluidic systems) where the DNA strands are allowed to hybridize. The Oligos are then ligated together (at 870). Finally, the assembled products may be purified from partial assembled products and left over free oligos (at 880).

Returning to FIGS. 3B and 3C, a second workflow of a second method of generating a longer DNA strand is provided. This method includes a two-step process for the generation of the final DNA strand: the first step includes multiple isolated reactions to generate different intron-exon strands, and the second step is to combine these different intron-exon strands into a final DNA strand. While on its face this two-stage process may appear more complex than the prior disclosed DNAzyme aided DNA strand synthesis technique, the benefit of this two-stage process is the ability to utilize fewer specialty designed DNAzyme molecules. This advantage is significant in that the design and synthesis of unique DNAzyme molecules is more difficult than the design and synthesis of intron and exon/functional oligonucleotide strands (which require specialty designs and synthesis regardless).

In this example process, as seen in FIG. 3B, an “exon-half-intron complex” or functionalized oligonucleotide complex 322 is generated. This exon-half-intron complex 322 includes an encoded region 326 surrounded by regions of half-introns 324 and 326 respectively. These half-intron regions are designed to couple to one end of standardized DNAzymes 342 and 352 respectively. The other end of the respective DNAzymes 342 and 352 is designed to couple to a left half-intron 312 and a right half-intron 332 respectively. These half-introns 312 and 332 include two portions. Labeled here, the left half-intron 312 includes a ‘middle’ region 314 and a ‘tail’ region 316. The right half-intron 332 includes a tail region 334 and a middle region 336.

When the DNAzymes 342 and 352, each half-intron 312 and 332, and the exon-half-intron complex 322 are functionalized (not illustrated) and placed in solution with one another, they couple together in a known/intended sequence. It can be seen that the tail 316 and middle 314 of the left half-intron 312 couple to one end of the first DNAzyme 342. The ‘left’ side half intron 324 of the exon-half-intron complex 322 couples to the other side of the first DNAzyme 342. The ‘right’ side half intron 328 of the exon-half-intron complex 322 couples to the first side of the second DNAzyme 352 and the right half-intron 332 couples to the other side of the second DNAzyme 352. A ligation process occurs, and the exon-intron DNA strand 362 is produced. This exon-intron DNA strand 362 is comprised of two full-introns surrounding the functional oligonucleotide segment 326. The ‘left’ intron is comprised of segments 314, 316 and 324. The ‘right’ intron is comprised of segments 328, 334 and 336. This process may be repeated many times, with different half-intron sequences 312 and 332, and different functional oligonucleotide sequences 326 to generate many different exon-intron DNA strands. This process, while requiring different DNAzymes 342 and 352, requires fewer types of DNAzymes varieties, and therefore may be standardized (as opposed to requiring uniquely designed DNAzymes every reaction).

The second step of this two-stage process is illustrated at FIG. 3C. Here the various exon-intron DNA strands 362, 372 and 374 have been synthesized as previously discussed in relation to FIG. 3B. Each exon-intron DNA strand 362, 372 and 374 includes an encoded region in the middle, surrounded by introns. While only three exon-intron DNA strands 362, 372 and 374 are illustrated here for the sake of clarity, it is understood that a very large number of exon-intron DNA strands may be employed at this step.

Standardized unique DNAzymes 382 and 384 are introduced into solution with the activated (not illustrated) exon-intron DNA strands 362, 372 and 374. These couple to the exon-intron DNA strands 362, 372 and 374 and assist in the ligation of the exon-intron DNA strands 362, 372 and 374 to generate a long final DNA strand 392.

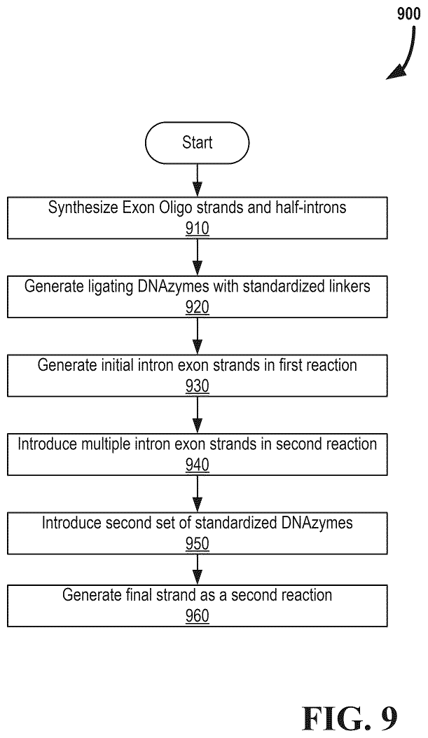

Turning now it FIG. 9, an example process for this two-stage methodology of final DNA strand generation is provided, seen generally at 900. In this method, the half intron with a middle encoding exon is first generated (at 910) along with the half-introns. As with other DNAzyme processes, the half-introns and the exon-half-intron segments are functionalized/activated (not illustrated). As previously noted, the ‘ends’ of the exon-half-intron segments correspond to standardized DNAzymes, which are likewise generated or otherwise procured (at 920). The DNAzymes, the exon-half-intron segments and the pair of half-introns are introduced to one another in solution. The half-introns, couple to one end of the corresponding DNAzyme, while the intron portions of the exon-half-intron strands couple to the other end of the DNAzymes, respectively. Due to the sequences of the half-intron segments, and the end portions of the exon-half-intron segments the coupling results in a particular order of the exon-half-intron segments and half-intron segments. In the presence of zinc, or other suitable metal, the DNAzyme causes the exon-half-intron segments and half-intron segments to ligate in the appropriate order (at 930). This may be performed repeatedly with different strand sequences in order to generate many different full intron-exon DNA strands. In the second stage of the process, these intron-exon DNA strands are introduced to a second reaction solution (at 940). Again, each intron-exon DNA strand is functionalized/activated, and a set of standardized DNAzymes that correspond to the ends of the intron-exon DNA strands is introduced (at 950). These DNAzymes are have unique ends. The ends of the intron-exon DNA strands may be particularly designed to ensure that, when placed in solution with the standardized DNAzymes, couple in a desired order. The various ordered intron-exon DNA strands are then ligated, as previously covered in considerable detail, to generate a final DNA strand (at 960).

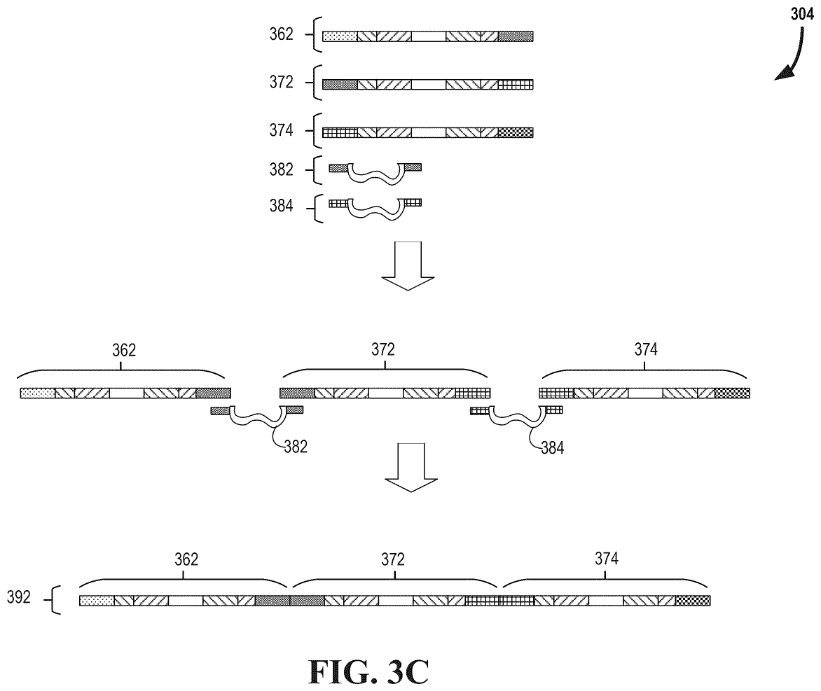

Now that a novel methodology of generating oligonucleotide ssDNA assemblies has been discussed, attention will be focused upon the ability to use target ssDNA segments to query a DNA library/database and/or determine if a given DNA segment is present. These techniques rely upon a phenomenon known as Förster Resonance Energy Transfer (FRET). FIG. 4 provides the basics of the FRET phenomenon. In this example, there are two molecules present. One is referred to as CFP (a doner fluorophore) 430 and the other YEP (an acceptor fluorophore) 440. These fluorophores are bombarded with electromagnetic radiation in a particular wavelength. The radiation is absorbed by the donor fluorophore causing it to become excited and then releasing/emitting radiation in a different wavelength from the absorbed radiation, as seen at 410. The acceptor fluorophore 440 is not-reactive to the bombarded radiation wavelength.

When the donor 430 and acceptor 440 fluorophores are relatively distant from one another, as in 410, there is no interaction between these two molecules. However, when these molecules are brought into very close proximity, the emissions from the donor fluorophore 430 may excite the acceptor fluorophore 440, resulting in yet another emission of another wavelength, as seen at 420. This emission by the acceptor fluorophore 440 may be measured using a photodetector, and thus, it can be determined when the two molecules are pulled close to one another.

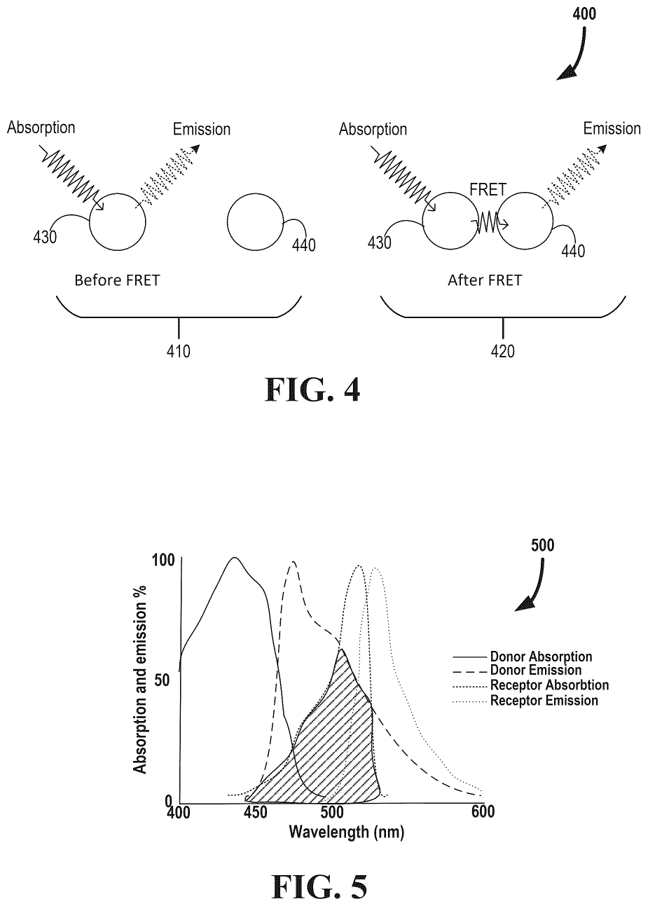

FIG. 5 provides an example of the radiation absorption and emission of the two fluorophores to better illustrate the FRET process. The absorption spectrum of the donor fluorophore 430 is centered around roughly 430 nm, in this particular example. Thus, the sample may be bombarded by light in this approximate wavelength. The emission by the donor fluorophore 430 centers around 475 nm, but extends to 515 nm before tapering off significantly. This is important because the absorption wavelength of the acceptor fluorophore 440 is centered on roughly 520 nm, so there is significant overlap between the emission of the donor and the absorption of the acceptor (shown as the shaded portion of the curves). When excited in this way, the acceptor fluorophore 440 emits light at approximately 525 nm. A photodetector calibrated to measure light in this approximate wavelength is thus able to determine if, and where, these two fluorophores are in extremely close proximity to one another.

This phenomenon may thus be leveraged by a synthetically generated strand of ssDNA 605 to determine when it is in the proximity of a target/query ssDNA strand 615.

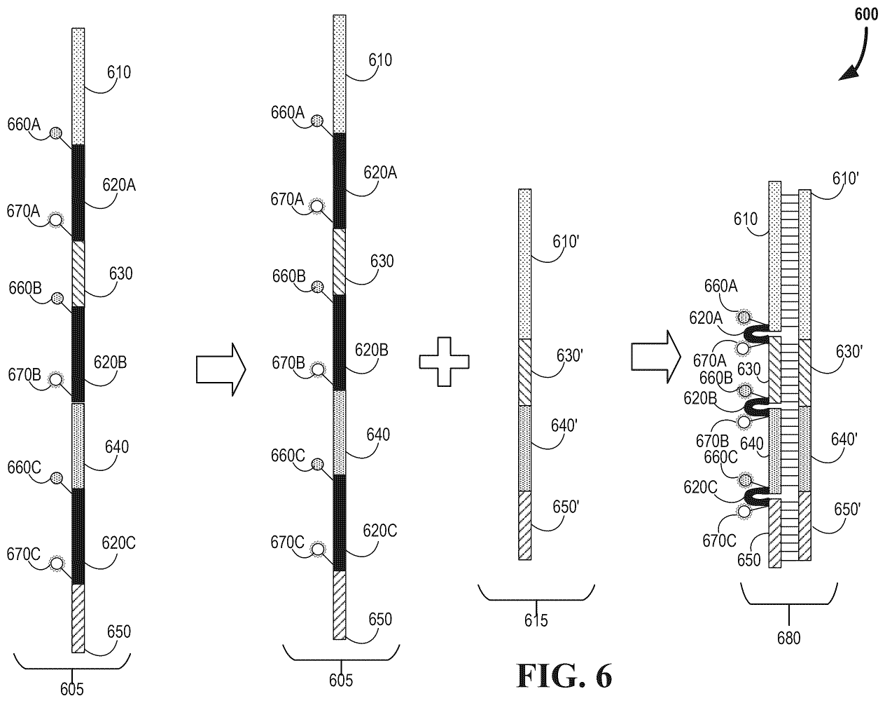

These DNA strands may be generated using any known techniques, but in some embodiments, the afore discusses DNAzyme enabled techniques may be particularly well suited for the generation of these library ssDNA segments 605. FIG. 6 provides greater detail into how the DNA query works, as seen at 600. The ‘library’ ssDNA 605 is composed of ‘query’ segments, which are oligo portions of DNA that are of interest. These query segments are shown here as 610, 630, 640 and 650 respectively. In between the query segments of the library ssDNA segment 605 are regions of inactive nucleotide regions (known as introns) 620A-C. These introns may be the same as one another or may be different. The important part is that these introns do not correspond to any known query DNA sequence. In some embodiments, the introns may be composed on all one base pair type, for example. On the ends of each intron may be a donor fluorophore 670A-C, and on the other end an acceptor fluorophore 660A-C. The introns may be of sufficient length (e.g., 20-50 base pairs in length) in order to not have the donor fluorophore 670A-C interact with its respective acceptor fluorophore 660A-C. In some embodiments, a phosphorescent or other non-fluorescent donor chromophore may be used instead of a fluorophore to prevent photobleaching of the acceptor chromophores. In yet other embodiments, the chromophores (donor fluorophore 670A-C and acceptor fluorophore 660A-C) may be fluorophores such as small organic dyes, quantum dots, or fluorescent proteins.

A query ssDNA segment 615 is introduced to the library ssDNA segment 605. The query ssDNA segment 615 includes complementary sequences of nucleotides to the query sequences. The result of these two pieces of ssDNA being introduced to one another is the hybridization of the non-intron oligo segments 610, 630, 640 and 650 of the database ssDNA segment 605 with their complementary regions 610′, 630′, 640′ and 650′ on the query ssDNA sequence 615. This results in a hybridized double stranded DNA segment 680 where the introns 620A-C are compressed and loop back upon one another. This results in the fluorophore donor 670A-C and the fluorophore acceptor 660A-C being drawn close together. When this hybridized double stranded DNA segment 680 is then bombarded with radiation in the absorption range of the fluorophore donor 670A-C the FRET phenomena occurs and the fluorophore acceptor 660A-C will emit light at a detectible wavelength. In this manner, it can be determined when (and potentially where) a hybridization event is occurring. This is useful for querying if a specific segment of ssDNA is present in a sample (for pathogen identification for example).

The example illustrated in FIG. 6 includes a ‘full match’ between the database ssDNA strand 605 and the complimentary query ssDNA strand 615. It is also possible that only partial matches between the query and database strands occurs. In the event of a partial match, only a fraction of the intron regions will fold back upon themselves, which will result in a weaker detected FRET signal. The degree of FRET signal detected can provide some information as to the percentage match between any given query ssDNA strand 615 and a database ssDNA strand 605.

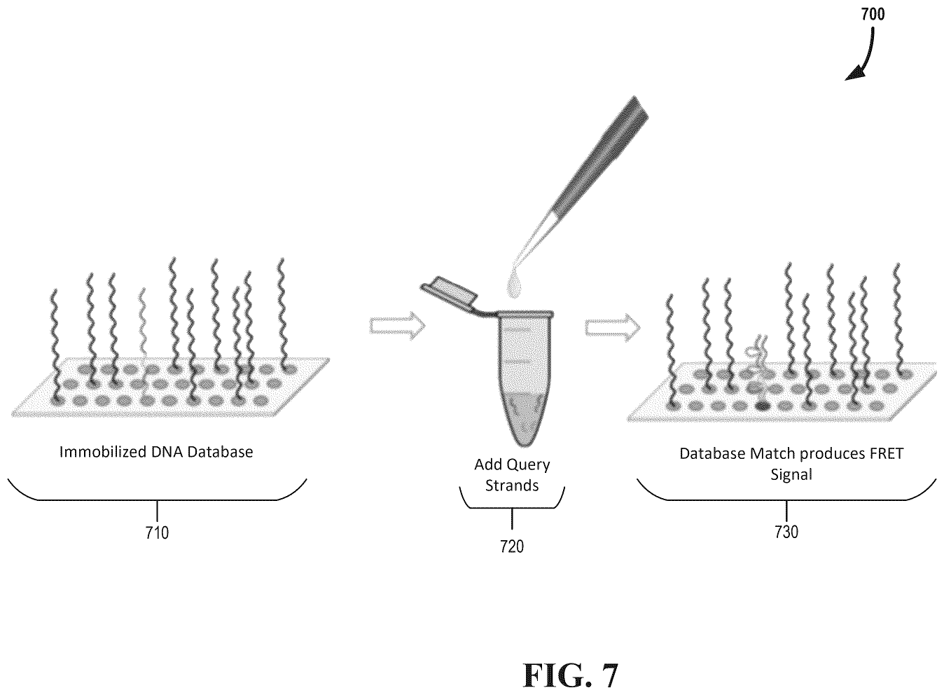

Turning to FIG. 7, the scalability of this FRET enabled technique for identifying matching database ssDNA segments 605 with query ssDNA segments 615 is illustrated. In this example, a very large number of database ssDNA segments are generated. These database ssDNA segments are unique from one another and may be deterministically located upon a substrate matrix, as seen in the complex 710. These database ssDNA segments may be generated using the DNAzyme techniques described previously.

In some embodiments, for example, the ssDNA database could be DNA fragments that correspond to different pathogens. Query strand(s) may be isolated from a sample, or may be artificially produced. In this example, assume the query strands are isolated from a medical sample, shown at 720. The isolated query strand is introduced to the database complex 710, and a hybridization occurs. This hybridized database 730 produces the detectible light emission due to the FRET phenomena at the location where the hybridization occurs. This allows the user to identify which sample on the matrix has been hybridized, and as these samples are deterministically placed, the ssDNA sequence of the query sample. In our example, assume the DNA database complex 710 is filled with different pathogen ssDNA segments. The segment for E. coli is different from the ssDNA strand for meningitis which in turn is different from the sample for strep throat and so on. When the query DNA strand is isolated from the test sample (e.g., a nasal swab, blood culture, etc.) it will hybridize with the complimentary ssDNA strand in the DNA database, resulting in the FRET phenomena, and therefore allowing the researcher/technician to rapidly identify which pathogen is being detected based upon the location of the light emission.

This DNA library query technique has obvious implications for medical diagnostics, but may also be helpful for medical research, DNA data storage, environmental research, sample isolation and any situation where it would be beneficial to determine what the sequence of a query DNA sample is from among a set of likely DNA sequences.

FIG. 9 provides a flow diagram 900 of the example process for the querying of a DNA library/database leveraging the FRET phenomena. Initially, the database needs to be generated (at 910) with ssDNA strands that include oligos of functional nucleotide sequences broken up by segments of introns. These database ssDNA strands may be deterministically immobilized on a substrate (e.g., silicon substrate, lab on a chip, etc.). Each database ssDNA strand may be unique from one another, and include different nucleotide sequences for the functional oligos.

The introns include a donor fluorophore on one end of the intron (3′ for example), and an acceptor fluorophore on the other end of the intron (5′ for example). The length of the intron must be sufficient to allow for easy looping of the intron when a hybridization event occurs, and of a sufficient length such that the donor fluorophore and the acceptor fluorophore do not exhibit FRET phenomena when the ssDNA database strand is not hybridized.

A query ssDNA strand, which is complementary to the functional oligo portions of one of the database ssDNA strands, is isolated or otherwise generated. This query ssDNA strand is introduced to the DNA database using wet chemistry (at 920). The query ssDNA strand hybridizes with its complementary database ssDNA strand, resulting in fluorescence due to the FRET phenomena as previously discussed in considerable detail. This fluorescence is measured (at 930) using a photodetector. This provides information as to where within the DNA database matrix the hybridization has occurred (at 940). Since the sequences of the ssDNA database strands are known, as well as their locations within the DNA database matrix, this fluorescence identifies the sequence of the query ssDNA strand.

Querying a DNA database in this manner can be much faster than querying a traditional database. It also removes the need for sequencing DNA, since the locations of the ‘files’ in each database are known. In the case of patient sample testing, critical time may be saved and effective treatments quickly identified.

DNA databases can be entirely synthetic sequences that encode data.

Alternatively, the DNA database may include pathogen or other database ssDNA strands for the purpose of disease identification. Similarly, the DNA database can also be genomic sequences that are synthesized gene-by-gene. These types of genome databases can have many applications. An example application can be used to find effective antibiotics to treat bacterial infections. A bacterium can be identified by its 16S gene, which is unique to each bacterium. Bacteria frequently acquire antibiotic resistance genes which confer resistance to certain antibiotics. A technician can test samples immediately for antibiotic resistance by adding the patient sample to a second database containing all known antibiotic resistance genes. Any matches will indicate which antibiotics the bacteria is resistant to.

While this invention has been described in terms of several embodiments, there are alterations, modifications, permutations, and substitute equivalents, which fall within the scope of this invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method for querying a DNA database comprising:
generating at least one database single stranded DNA (ssDNA) strand, wherein the database ssDNA strand is comprised of a plurality of functional oligonucleotides separated by introns, and wherein each intron includes a 3′ end and a 5′ end, and wherein each intron includes a donor fluorophore on one end and an acceptor fluorophore on the other end.

2. The method of claim 1, further comprising generating a query ssDNA strand, wherein the query ssDNA stand comprises a series of complimentary oligonucleotides, which are capable of hybridizing with the functional oligonucleotides of at least one of the at least one database ssDNA strand.

3. The method of claim 2, wherein the query ssDNA is isolated from a biological sample.

4. The method of claim 2, wherein the query ssDNA is synthetically derived.

5. The method of claim 2, further comprising introducing the query ssDNA strand to the at least one database ssDNA strand, wherein the query ssDNA strand hybridizes with at least one of the at least one database ssDNA strand, and wherein the hybridization causes the intron regions of the at least one of the at least one database ssDNA strand to fold resulting in the donor fluorophore on one end and the acceptor fluorophore on the other end of each intron to be placed in close proximity to one another.

6. The method of claim 5, wherein the close proximity of the donor and acceptor fluorophores enables Förster Resonance Energy Transfer (FRET) phenomena to occur at each intron.

7. The method of claim 6, wherein the emission of the FRET phenomena is detected using a photodetector.

8. The method of claim 7, wherein the at least one database ssDNA strand includes a plurality of database ssDNA strands, and wherein a subset of the plurality of database ssDNA strands is unique from another.

9. The method of claim 8, further comprising immobilizing the plurality of database ssDNA strands on a substrate, wherein the sequence for each database ssDNA strand is known, and the location on the substrate of a cluster of copies of the database ssDNA strand is known.

10. The method of claim 9, wherein the detecting the FRET phenomena enables determining which database ssDNA strand of the plurality of ssDNA strands immobilized on the substrate has hybridized with the query ssDNA strand, thereby determining the sequence of the query ssDNA strand.

11. The method of claim 10, wherein the plurality of database ssDNA strands each correspond to a different pathogen unique DNA or RNA.

12. The method of claim 11, further comprising determining which pathogen the query ssDNA strand is derived from based upon the known sequence.

13. The method of claim 10, wherein the plurality of database ssDNA strands each correspond to a different antibiotic resistance DNA sequence.

14. The method of claim 13, further comprising determining which antibiotics a pathogen the query ssDNA strand is derived from is resistant to based upon the known sequence.

15. A DNA database system for querying by a query single stranded DNA (ssDNA) strand, the system comprising:
a substrate; and
a plurality of database ssDNA strands immobilized on the substrate, wherein each database ssDNA strand is comprised of a plurality of functional oligonucleotides separated by introns, and wherein each intron includes a 3′ end and a 5′ end, and wherein each intron includes a donor fluorophore on one end and an acceptor fluorophore on the other end.

16. The system of claim 15, wherein the each of the plurality of database ssDNA is capable of hybridizing with a different query ssDNA strand, wherein the hybridization causes the intron regions of the hybridized database ssDNA strand to fold resulting in the donor fluorophore on one end and the acceptor fluorophore on the other end of each intron to be placed in close proximity to one another which enables Förster Resonance Energy Transfer (FRET) phenomena to occur at each intron.

17. The system of claim 16, further comprising a photodetector configured to detect the presence of and location of a FRET occurrence.

18. The system of claim 17, and wherein each of the plurality of database ssDNA strands is unique from another, wherein the sequence for each database ssDNA strand is known, and wherein the location on the substrate of each database ssDNA strand is known.

19. The method of claim 18, wherein the detecting the FRET phenomena enables determining which database ssDNA strand of the plurality of ssDNA strands immobilized on the substrate has hybridized with the query ssDNA strand, thereby determining the sequence of the query ssDNA strand.

20. A method for generating a database single stranded DNA (ssDNA) strand comprising:
receiving a plurality of functional oligonucleotides, wherein each of end of the functional oligonucleotides include a unique sequence of base pairs that are designed;
receiving plurality of introns, wherein each of end of the introns include a unique sequence of base pairs that are designed, and wherein each intron includes a donor fluorophore on one end and an acceptor fluorophore on the other end; and
introducing a plurality of DNAzymes to the plurality of functional oligonucleotides and the plurality of introns, wherein the DNAzymes are designed to have ends that are of a known sequence, and wherein the designing the ends of the plurality of functional oligonucleotides, the plurality of introns, and the DNAzymes results in conjugation of the plurality of functional oligonucleotides and the plurality of introns in a desired order.

21. A method for generating a database single stranded DNA (ssDNA) strand comprising the steps of:
a. receiving a plurality of functional oligonucleotides, wherein each of end of the functional oligonucleotides include a unique standardized sequence of base pairs;
b. receiving plurality of a pair of half introns, wherein each of the half introns include a first section of unique sequence of base pairs and a second section of unique base pairs, and wherein a first of the pair of half introns and a second of the pair of half introns include different unique sequences;
c. introducing a plurality of a pair of DNAzymes to the plurality of functional oligonucleotides and the plurality of the pair of half introns, wherein the DNAzymes are designed to have ends that are of a known sequence, and wherein the designing the ends of the plurality of functional oligonucleotides, the plurality of half introns, and the DNAzymes results in conjugation of the plurality of functional oligonucleotides and the plurality of half introns in a desired order;
d. ligating each of a subset of the plurality of functional oligonucleotides with the first half intron on a 3′ end of each functional oligonucleotide and the second half intron on a 5′ end of the functional oligonucleotide to generate a plurality of half intron-exon strands;
e. repeating steps a-d with different unique sequences of base pairs to generate a plurality of different half intron-exon strands;
f. introducing a plurality of DNAzymes that are unique from the pair of DNAzymes, wherein the plurality of DNAzymes are standardized, and wherein the plurality of DNAzymes conjugate with the plurality of different half intron-exon strands in a desired order; and
g. ligating the plurality of different half intron-exon strands in the desired order to generate a plurality of final ssDNA strands.
